(19) United States
(12) Patent Application Publication
(10) Pub. No.: US 2022/0241377 A1
(43) Pub. Date: Aug. 4, 2022

(54) RECEPTOR-TARGETING PEPTIDE-DRUG CONJUGATES

(71) Applicant: MOLGENIE GMBH, Stuttgart (DE)

(72) Inventors: Wolfgang Richter, Munchen (DE); Lutz Weber, Stuttgart (DE); Robert Rennert, Halle/Saale (DE)

(21) Appl. No.: 17/629,460

(22) PCT Filed: Jul. 24, 2020
(86) PCT No.: PCT/EP2020/070985

§ 371 Date: Jan. 24, 2022

(30) Foreign Application Priority Data
Jul. 24, 2019 (EP) 19188092.1

(51) Int. Cl. A61K 38/22; C07K 14/575; A61K 47/64; A61K 47/54; A61P 35/00

(52) U.S. Cl. CPC A61K 38/2271; C07K 14/57545; A61P 35/00; A61K 47/542; A61K 47/64

(57) ABSTRACT

The present invention refers to NPY Y1 receptor-targeting peptide moieties, and their use for the target-specific treatment of cancers and other diseases.

The present invention refers to NPY Y1 receptor-targeting peptide moieties, and their use for the target-specific treatment of cancers and other diseases.

It is an object of the present invention to provide novel artificially modified receptor target-specific peptides that are suitable to be used as targeting moiety in peptide-drug conjugates (PDCs) that are composed—besides this peptidic cell surface receptor ligand—of at least one pharmacologically active molecule that is coupled to the peptide moiety via a suitable chemical linker structure. These PDCs are intended to address cancer cells that express, ideally overexpress, the GPCR family of human neuropeptide Y (NPY) receptors, especially the NPY Y1 receptor subtype (hY1R). Thereby the described PDCs are necessarily composed of one of the novel artificially modified peptides described herein that is an agonistic ligand of the NPY Y1 receptor, and a highly potent, e.g. cytotoxic, cytostatic, pro-apoptotic, anti-angiogenic etc., compound derivative (therapeutic payload) that is coupled to the peptide moiety via a suitable, ideally chemically (e.g. pH, redox etc.) or metabolically (e.g. enzymatically) cleavable chemical linker structure, thereby providing the cancer cell-selective targeting property of the hY1R-specific peptide moiety to enhance the cancer selectivity of the treatment and the therapeutic window of the highly potent therapeutic payload.

Importantly, the present invention describes novel artificially modified peptides, and appropriate PDCs comprising those peptide moieties, that are derived from pig NPY (pNPY), but include several amino acid modifications that were unexpectedly found to yield high selectivity for the human NPY Y1 receptor (hY1R), and, most importantly, differ strongly in the C-terminal peptide part, namely in the amino acid positions 33-36, from wild type NPY (wild type: -Arg³³-Gln³⁴-Arg³⁵-Tyr³⁶-amide). Furthermore, the peptides described herein contain further sequence modifications and sequence branching.

It has been shown that the hY1R is overexpressed in several cancer types, such as breast cancers of all major breast cancer types (i.e. hormone receptor positives, HER2/neu positives, and triple-negatives; Poster 1745 at the annual AACR meeting, Philadelphia, 2015) and especially even metastatic breast cancer (Reubi J. C. et al., Cancer Res. 2001, 61: 4636-4641). Beyond breast cancers, hY1R overexpression was also detected in other cancer conditions, particularly in Ewing's sarcoma, Synovial sarcoma and Leiomyosarcoma (Körner et al., Clin. Cancer Res. 2008, 14: 5043-5049), but also renal cell carcinomas and nephroblastomas (Körner M. et al., Int. J. Cancer 2005, 115: 734-741), neuroblastic tumors, paragangliomas, pheochromocytomas and adrenal cortical tumors (Körner M. et al., Clin. Cancer Res. 2004, 10: 8426-8433), ovarian sex cord-stromal tumors and ovarian adenocarcinomas (Körner M. et al., Lab. Investigation 2004, 84: 71-80; Körner and Reubi, Peptides 2007, 28: 419-425).

Several therapeutic NPY-derived peptide-drug conjugates have been tested and published. However, none of these NPY-based conjugates proved a convincing in vivo efficacy. For example, IPSEN Pharma SAS claimed PDCs for NPY receptor targeting containing, for instance, paclitaxel, doxorubicin or camptothecin coupled to the peptide moiety by covalent amino acid linkers (PCT/US2010/000473). The patent application comprises MCF-7 xenograft in vivo data for three PDCs, whereby the best of these compounds caused significant effects just at a dose >100 mg/kg, what is doubtless too high for a competitive therapy option.

Furthermore, two previous patent applications of OntoChem GmbH deal with receptor ligand-linked cytotoxic molecules that are based on [F⁷,P³⁴]-pNPY derived peptide analogues and comprise cleavable linker structures and various cytotoxic payloads, such as tubulysins amongst others (PCT/EP2013/002790) or monomethyl auristatines (PCT/EP2015/000558). However, even though the in vitro data of the PDCs claimed herein are promising and the in vivo efficacies (significant anti-tumor efficacies in tumor cell line-derived mouse xenograft models using doses <10 mg/kg) are better than the efficacies of the IPSEN Pharma SAS conjugates, the hY1R-targeting peptide-toxin conjugates are probably not potent enough to become clinical therapeutics. However, all published studies as well as all patents claiming hY1R-targeting peptide-drug conjugates are dealing exclusively with either wild type NPY peptide moieties or modified NPY peptide moieties with C-termini relatively close related to the wild type NPY C-terminus (-Arg³³-G1n³⁴-Arg³⁵-Tyr³⁶-amide), particularly the most prominent hY1R-selective [F⁷,P³⁴]-NPY or modified variants thereof.

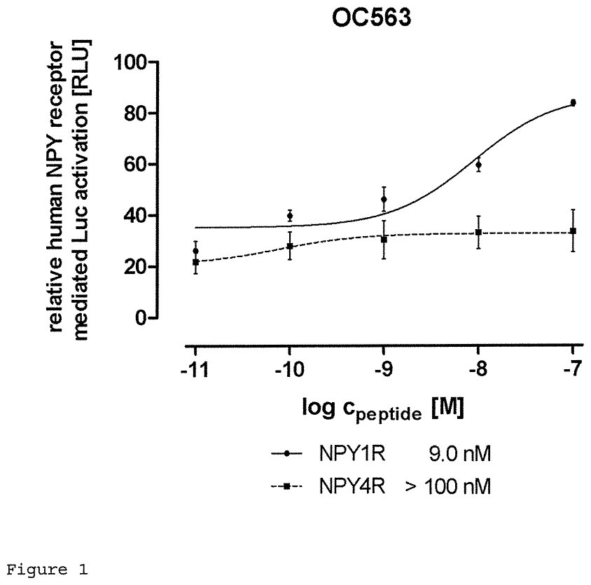

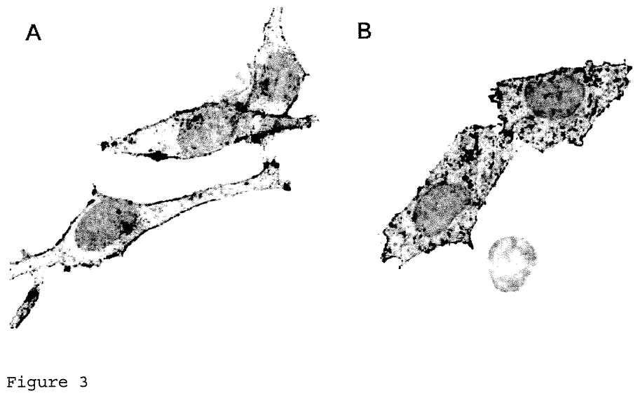

Unexpectedly, it has been found that novel artificially modified peptides that are based on the well-established [F⁷,P³⁴]-pNPY, but wherein the C-terminal positions 33, 35 and/or 36 (Arg³³, Arg³⁵ and Tyr³⁶) are replaced by alternative amino acids (for instance alanines such as Arg33Ala, Arg35Ala and Tyr36Ala), as well as peptide-toxin conjugates (PDCs) comprising these peptide moieties, exhibit surprisingly good functional hY1R activation and hY1R-mediated internalization in vitro (see below Examples and FIGS. 1 and 3).

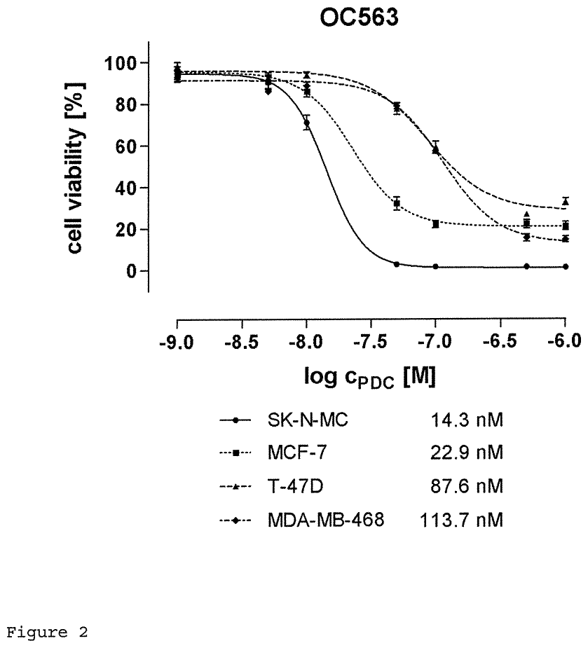

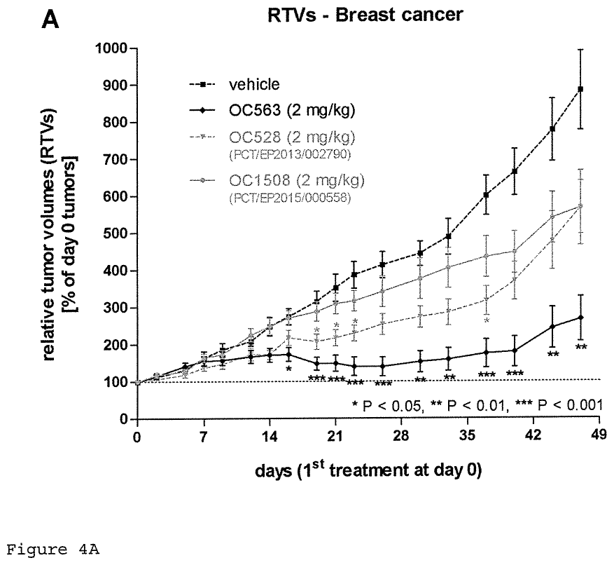

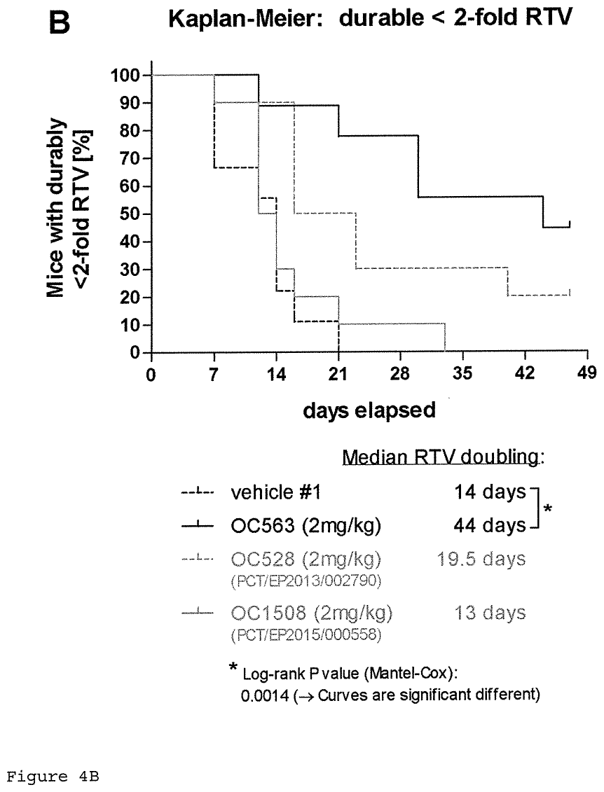

Even more surprisingly, PDCs comprising one of these novel artificially modified peptide moieties with its strongly atypical C-terminus permitted in vitro anti-tumor efficacies with IC₅₀ values in the low nanomolar range (see below Examples and FIG. 2) and potent in vivo anti-tumor efficacy in a patient-derived breast cancer xenograft (breast cancer PDX) as well (Examples and FIG. 4A and 4B). Most surprisingly, and contrary to all so far established knowledge on prerequisites for a potent hY1R-addressing peptide, PDCs comprising one of these novel artificially modified peptide moieties with its strongly atypical C-terminus, for instance containing Ala³³, Ala³⁵ and Ala³⁵, were significantly more effective in the breast cancer PDX animal models than PDCs containing the well-established “gold standard” of highly affine hY1R-selective peptides, [F⁷,P³⁴]-pNPY (see below in FIGS. 4A and 4B wherein the novel, herein claimed conjugate OC563 is compared with the recently claimed OC528 and OC1508; PCT/EP2013/002790 and PCT/EP2015/000558).

The present invention provides compounds having the following formula (I):

R¹-Tyr¹-Pro²-Ser³-Lys⁴-Pros-Asp⁶-Phe⁷-Pro⁸-Gly⁵⁻Glu¹⁰⁻Asp¹¹⁻Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Xaa³³-Pro³⁴-Xaa³⁵-Xaa³⁶-NH₂ (I)

wherein
R¹ is hydrogen or an acyl group;
Xaa³³ is Arg or a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is Arg or a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1; and
Xaa³⁶ is Tyr or a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1;
with the proviso that Xaa³³ is not Arg, when Xaa³⁵ is Arg and Xaa³⁶ is Tyr;
or a salt thereof.

The present invention further provides compounds having the following formula (I):

R¹-Tyr¹-Pro²-Ser³-Lys⁴-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Xaa³³-Pro³⁴-Xaa³⁵-Xaa³⁶-NH₂ (I)

wherein
R¹ is hydrogen or an acyl group;
Xaa³³ is a group of formula —N(R²)—CH(R³)—(CH₂)ₙ—(═O)—, wherein R² is hydrogen or a methyl group, R³ is hydrogen or a linear or branched C₁₋₈ alkyl group and n is 0 or 1;
Xaa³⁵ is a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1; and
Xaa³⁶ is a group of formula —N(R⁸)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1;
or a salt thereof.

The present invention moreover provides compounds having the following formula (II):

R¹-Pro²-Ser³-Lys⁴ (R⁸)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Xaa³³-Pro³⁴-Xaa³⁵-Xaa³⁶-NH₂ (II)

wherein
R¹ is hydrogen or an acyl group;
Xaa³³ is Arg or a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is Arg or a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1;
Xaa³⁶ is Tyr or a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1;
and
R⁸ is bound to the nitrogen atom at the side chain of the lysine (Nε) and is selected from the following groups: R⁹-Cys- and R⁹-Cys-βAla-, wherein R⁹ is hydrogen or an acyl group;
with the proviso that Xaa³³ is not Arg, when Xaa³⁵ is Arg and Xaa³⁶ is Tyr;
or a salt thereof.

The present invention further provides compounds having the following formula (II):

R¹-Pro²-Ser³-Lys⁴ (R⁸)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Xaa³³-Pro³⁴-Xaa³⁵-Xaa³⁶-NH₂ (II)

wherein
R¹ is hydrogen or an acyl group;
Xaa³³ is a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is a group of formula —N(R⁴)—CH(R⁶)—(CH₂)ₘ—C(═O)—, wherein R⁴ is hydrogen or a methyl group, R⁵ is hydrogen or a linear or branched C₁₋₈ alkyl group and m is 0 or 1;
Xaa³⁶ is a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1;
and
R⁸ is bound to the nitrogen atom at the side chain of the lysine (Nε) and is selected from the following groups: R⁹-Cys- and R⁹-Cys-βAla-, wherein R⁹ is hydrogen or an acyl group;
or a salt thereof.

Preferably, R¹ is hydrogen or an acetyl group.

Further preferably, Xaa³³ is selected from alanine (Ala; A), valine (Val; V), leucine (Leu; L), isoleucine (Ile; I), beta-alanine (βAla; (βA), N-methyl-alanine (N-Me-Ala), norvaline (Nva), norleucine (Nle), β-homo-leucine (β-homo-Leu), β-homo-isoleucine (β-homo-Ile), N-methyl-isoleucine (N-Me-Ile), and N-methyl-norleucine (N-Me-Nle); especially preferably, Xaa³³ is Ala.

Moreover preferably, Xaa³⁵ is selected from alanine (Ala; A), valine (Val; V), leucine (Leu; L), isoleucine (Ile; I), beta-alanine (βAla; βA), N-methyl-alanine (N-Me-Ala), norvaline (Nva), norleucine (Nle), β-homo-leucine (β-homo-Leu), β-homo-isoleucine (β-homo-Ile), N-methyl-isoleucine (N-Me-Ile), and N-methyl-norleucine (N-Me-Nle); especially preferably, Xaa³⁵ is Ala.

Further preferably, Xaa³⁶ is selected from alanine (Ala; A), valine (Val; V), leucine (Leu; L), isoleucine (Ile; I), beta-alanine (βAla; βA), N-methyl-alanine (N-Me-Ala), norvaline (Nva), norleucine (Nle), β-homo-leucine (β-homo-Leu), β-homo-isoleucine (β-homo-Ile), N-methyl-isoleucine (N-Me-Ile), and N-methyl-norleucine (N-Me-Nle); especially preferably, Xaa³⁶ is Ala.

Moreover preferably, R⁹ is selected from the following groups: palmitoyl, tetradecanoyl, dodecanoyl, decanoyl, octadecanoyl or acetyl; preferably from palmitoyl and dodecanoyl; especially preferably, R⁹ is palmitoyl.

Especially preferred are the following compounds:

H-Tyr¹-Pro²-Ser³-Lys⁴-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Ala³³-Pro³⁴-Ala³⁵-Ala³⁶-NH₂;
Acetyl-Tyr¹-Pro²-Ser³-Lys⁴-Pro⁵-Asp⁶-Phe⁷-Pro⁸-Gly⁹-Glu¹⁰-Asp¹¹-Ala¹²-Pro¹³-Ala¹⁴-Glu15-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Ala³³-Pro³⁴-Ala³⁵-Ala³⁶-NH₂;
H-Tyr¹-Pro²-Ser³-Lys⁴(Palmitoyl-Cys-βAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁸-His²⁶-Tyr²⁷-Ile²⁸-Asn²⁹-Leu³⁰-Il³¹-Thr³²-Ala³³-Pro³⁴-Ala³⁵-Ala³⁶-NH₂;

Acetyl-Tyr¹-Pro²-Ser³-Lys⁴ (Palmitoyl-Cys-βAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Ala³³-Pro³⁴-Ala³⁵-Ala³⁶-NH₂;

or a salt thereof.

The present invention further provides compounds of formula (III):

Pep-L-Z (III)

wherein
Pep is a compound of formula (II′)

R¹-Tyr¹-Pro²-Ser³-Lys⁴(R⁸)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Xaa³³-Pro³⁴-Xaa³⁵-Xaa³⁶-NH₂ (II′)

wherein
R¹ is hydrogen or an acyl group;
Xaa³³ is Arg or a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is Arg or a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1;
Xaa³⁶ is Tyr or a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1;
with the proviso that Xaa³³ is not Arg, when Xaa³⁵ is Arg and Xaa³⁶ is Tyr;
and
R⁸ is bound to the nitrogen atom at the side chain of the lysine (Ne) and is selected from the following groups: R⁹-Cys- and R⁹-Cys-βAla-, wherein R⁹ is hydrogen or an acyl group;
wherein the hydrogen atom at the SH moiety of Cys at group R⁸ is replaced by the bond to L;
L is a linker between Pep and Z; and
Z is a natural or synthetic tubulysin derivative wherein one hydrogen atom or one OH group has been replaced by the bond to L;
or a salt thereof.

The present invention moreover provides compounds of formula (III):

Pep-L-Z (III)

wherein
Pep is a compound of formula (II′)

R¹-Tyr¹-Pro²-Ser³-Lys⁴ (R⁸)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Xaa³³-Pro³⁴-Xaa³⁵-Xaa³⁶-NH₂ (II′)

wherein
R¹ is hydrogen or an acyl group;
Xaa³³ is a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1;
Xaa³⁶ is a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1;
and
R⁸ is bound to the nitrogen atom at the side chain of the lysine (NE) and is selected from the following groups: R⁹-Cys- and R⁹-Cys-βAla-, wherein R⁹ is hydrogen or an acyl group;
wherein the hydrogen atom at the SH moiety of Cys at group R⁸ is replaced by the bond to L;
L is a linker between Pep and Z; and
Z is a natural or synthetic tubulysin derivative wherein one hydrogen atom or one OH group has been replaced by the bond to L;
or a salt thereof.

Preferably, L is selected from the following groups:

—CH₂—CH₂—S—;

—O—CH₂—CH₂—S—;

—NH—CH₂—CH₂—S—; or

—NH—NH—C(═O)—O—CH₂—CH₂—S—;

wherein the sulphur of L is bound to the sulphur of the Cys at group R⁸.

Especially preferably, L is a group of formula —NH—CH₂—CH₂—S—, wherein the sulphur of L is bound to the sulphur of the Cys at group R⁸.

Preferably, Z is a compound of formula (IV):

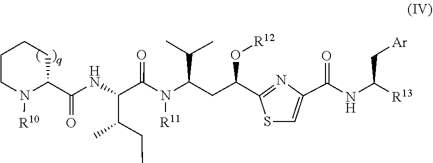

wherein
q is 0, 1 or 2;
R¹⁰ is an alkyl, acyl or a heteroalkyl group;
R¹¹ is an optionally substituted alkyl, alkenyl, alkinyl, acyl, heteroalkyl, cycloalkyl, heterocycloalkyl, alkylcycloalkyl, heteroalkylcycloalkyl, aryl, heteroaryl, aralkyl or heteroaralkyl group;
R¹² is hydrogen or an optionally substituted alkyl, alkenyl, alkinyl, acyl, heteroalkyl, cycloalkyl, heterocycloalkyl, alkylcycloalkyl, heteroalkylcycloalkyl, aryl, heteroaryl, aralkyl or heteroaralkyl group;
R¹³ is a group of formula —COOH, —CONH₂, —CONHNH₂ or —CH₂OH or a group of the following formula:

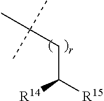

wherein r is 0 or 1; R¹⁴ is hydrogen or an optionally substituted C₁₋₆ alkyl group or an optionally substituted aryl or heteroaryl group; and R¹⁵ is a group of formula —COOH, —CONH₂, —CONHNH₂ or —CH₂OH; and
Ar is an optionally substituted arylene or heteroarylene group;
wherein one OH group of a COOH group or one hydrogen atom has been replaced by the bond to L.

Further preferably, Z has the following formula:

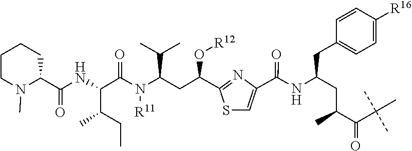

wherein
R¹¹ is hydrogen, a C₁₋₆ alkyl group, or a group of formula —CH₂—O—C(═O)—R¹⁷; wherein R¹⁷ is a C₁₋₆ alkyl group or a C₂₋₆ alkenyl group or an aryl group or a heteroaryl group;
R¹² is a C₁₋₆ alkyl group or an acetyl group; and
R¹⁶ is hydrogen, halogen, OH, NO₂, NH₂, CN, C₁₋₆ alkyl, —O—C₁₋₆ alkyl, phenyl, —NH—C₁₋₆ alkyl or —N(C₁₋₆ alkyl) ₂.

Moreover preferably, Z has the following formula:

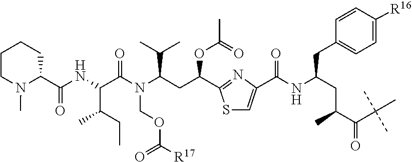

wherein R¹⁷ is hydrogen, or an alkyl, alkenyl, aryl or heteroaryl group and R¹⁶ is hydrogen or a hydroxy group.

Especially preferably, Z has the following formula:

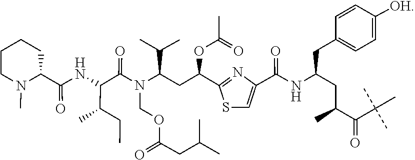

Further especially preferred are the following compounds:

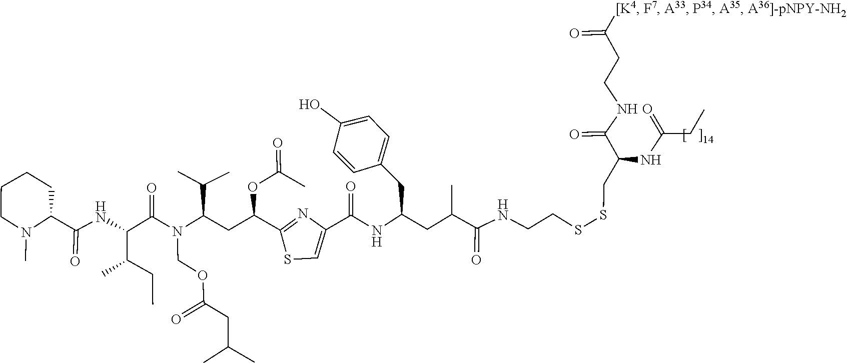

[K⁴ (Palmitoyl-C(Linker-TubA) -betaA) , F⁷, A³³, P³⁴, A³⁵, A³⁶]-pNPY-amide; and

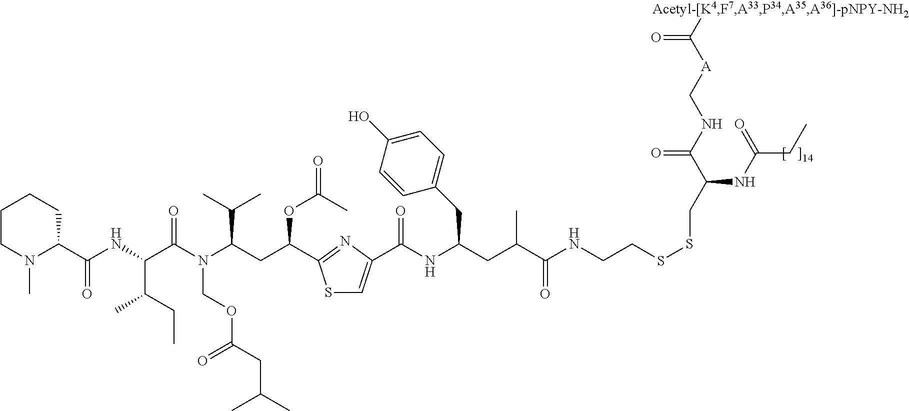

Acetyl-[K⁴ (Palmitoyl-C(Linker-TubA)-betaA) , F⁷, A³³, P³⁴, A³⁵, A³⁶]-pNPY-amide; or a salt thereof.

The present invention further relates to pharmaceutical compositions containing a compound of formula Pep-L-Z as described herein and optionally one or more carriers and/or adjuvants.

The present invention moreover relates to the use of a compound of formula Pep-L-Z or a pharmaceutical composition as described herein for the treatment of cancer.

The present invention further relates to the use of a compound of formula Pep-L-Z or a pharmaceutical composition as described herein for the preparation of a medicament for the treatment of cancer.

Moreover, the present invention relates to compounds or pharmaceutical compositions as described herein for use in the treatment of cancer.

The compounds described herein can comprise several chiral centers depending on their substitution pattern. The present invention relates to all defined enantio and diastereo isomers as well as their mixtures in all ratios. Moreover, the present invention relates to all cis/trans isomers of the compounds described herein as well as their mixtures. Moreover, the present invention relates to all tautomeric foLtus of the compounds described herein.

Examples of pharmacologically acceptable salts of the compounds described herein are physiologically acceptable mineral acids, e.g. hydrochloric acid, sulfuric acid, phosphoric acid or salts of organic acids, e.g. methansulfonic acid, p-toluenesulfonic acid, lactic acid, formic acid, trifluoracetic acid, citric acid, succinic acid, fumaric acid, maleic acid and salicylic acid. The compounds described herein can be solvated, especially hydrated. The hydration can occur during the synthesis process or can be a consequence of the hygroscopic nature of the originally dehydrated compounds described herein. As mentioned above, compounds described herein, containing asymmetric carbon atoms might exist as mixtures of diastereomers, as mixtures of enantiomers or as optically pure compounds.

Prodrugs are also subject of the present invention and they are composed of at least one compound described herein and at least one pharmacologically acceptable protecting group, which is cleaved under physiological conditions, e.g. alkoxy, aralkyloxy, acyl or acyloxy, more precisely ethoxy, benzyloxy, acetyl or acetyloxy.

The therapeutic use of the compounds described herein, their pharmacologic acceptable salts and/or solvates and hydrates, as well as the corresponding formulations and pharmacological compositions are also subject of the present invention.

Especially, the compounds described herein are of interest for the treatment of those cancer types with cancer-specific hY1R expression, particularly hY1R overexpression compared to the surrounding healthy tissues, such as breast cancers of all major breast cancer types (i.e. hormone receptor positives, HER2/neu positives, and triple-negatives), particularly also metastatic breast cancers, furthermore, various sarcoma cancer types like Ewing's sarcomas, Synovial sarcoma and Leiomyosarcoma, as well as, for instance, renal cell carcinomas, nephroblastomas, other neuroblastic tumors, paragangliomas, pheochromocytomas, adrenal cortical tumors, ovarian sex cord-stromal tumors, and ovarian adeno-carcinomas.

Furthermore, the compounds described herein may be used for treatment of any other cancer type, than the aforementioned cancer types, that is or will be characterized by hY1R expression, ideally hY1R overexpression compared to the surrounding healthy tissues.

In general, the compounds described herein can be given as a single treatment or as multiple treatments either alone or in combination with an arbitrary therapeutic substance according to known and accepted modes or as a continuous treatment whereby the active principle can be embedded in a matrix such as e.g. an implantable hydrogel.

Compositions according to the invention can be administered in one of the following ways: solutions, emulsions or suspensions; parenteral, including injectable solutions; by inhalation, including powder formulation or as a spray, transdeLmal or intranasal. For the production of liquid solutions and syrups one may use carriers for example water, alcohols, aqueous saline, aqueous dextrose, polyole, glycerin, vegetable oils, petroleum, animal or synthetic oils. For the production of suppositories one may use excipients like e.g. vegetable, petroleum, animal or synthetic oils, wax, fat and polyols. For aerosol formulations one may use compressed gases suitable for this purpose like e.g. oxygen, nitrogen, noble gas and carbon dioxide. The pharmaceutically useful agents may also contain additives for conservation, stabilization, e.g. UV stabilizer, emulsifier, sweetener, aromatiser, salts to change the osmotic pressure, buffers, coating additives and antioxidants.

Combinations with other therapeutic agents can include further agents, which are commonly used to treat the diseases mentioned above, especially cancers.

The term alkyl or alk refers to a saturated, linear or branched, optionally substituted hydrocarbon group, containing preferably from one to thirty, moreover preferably from one to twenty carbon atoms, further preferably from one to twelve carbon atoms, mostly preferred from one to six carbon atoms, for example methyl, ethyl, propyl, isopropyl, isobutyl, n-butyl, sek-butyl, tert-butyl, n-pentyl, 2,2dimethylpropyl, 2-methylbutyl, n-hexyl, 2,2-dimethylbutyl or 2,3-dimethylbutyl.

The term alkenyl and alkinyl refers to an at least partially unsaturated, linear or branched, optionally substituted hydrocarbon group, containing preferably from two to thirty, moreover preferably from two to twenty carbon atoms, further preferably from two to twelve carbon atoms, mostly preferred from two to six carbon atoms, for example ethenyl, allyl, acetylenyl, propargyl, isoprenyl, or hex-2-enyl. Preferentially, alkenyl groups contain one or two, most preferred one double bond and alkinyl groups contain one or two, most preferred one triple bond.

Optionally the terms alkyl, alkenyl and/or alkinyl refer to groups where one or several, preferentially one, two or three hydrogen atoms are replaced by a halogen atom, preferentially fluorine or chlorine or a 2,2,2-trichlorethyl, or a trifluoromethyl group.

The term heteroalkyl refers to an alkyl, alkenyl or alkinyl group, where one or more, preferentially one, two or three carbon atoms are replaced by an O, N, P, B, Se, Si, or S atom, preferentially O, S or N. The term heteroalkyl also refers to a carboxylic acid or a group derived thereof, for example acyl, acylalkyl, alkoxycarbonyl, acyloxy, acyloxyalkyl, carboxyalkylamid or alkoxycarbonyloxy.

Examples of heteroalkyl groups are groups of the formula R^{a}—O—Y^{a}, R^{a}—S—Y^{a}—R^{a}—N(R^{b})—Y^{a}—, R^{a}—CO—Y^{a}—, R^{a}—O—C—Y^{a}—R^{a}—C—O—Y^{a}—, R^{a}—CO—N (R^{b}—Y^{a}—, R^{a}—N—(R^{b})—CO—Y^{a}—, R^{a}—O—C—N(R^{b})—Y^{a}—, R^{a}—N(R^{b})—CO—O—Y^{a}—, R^{a}—N(R^{b})—CO—N(R^{c})—Y^{a}—, R^{a}—O—CO—O—Y^{a}—, R^{a}—N(R^{b})—C (═NR^{d})—N (R^{c})—Y^{a}—, R^{a}—CS—Y^{a}—R^{a}—O—CS—Y^{a}—, R^{a}—CS—O—Y^{a}—, R^{a}—CS—N(R^{b})—Y^{a}—, R^{a}—N—(R^{b})—CS—Y^{a}—R^{a}—O—CS—N(R^{b})—Y^{a}—, R^{a}—N(R^{b})—CS—O—Y^{a}—, R^{a}—N(^{R}b)—CS—N (R^{c})—Y^{a}—, R^{a}—O—CS—O—Y^{a}—, R^{a}—S—CO—Y^{a}—R^{a}—CO—S—Y^{a}—, R^{a}—S—CO—N(R^{b})—Y^{a}—, R^{a}—N (R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, wherein Ra refers to a H, a C₁-C₆-alkyl, a C₂-C₆-alkenyl or a C₂-C₆-alkinyl group; wherein R^{b} refers to a H, a C₁-C₆-alkyl, a C₂-C₆-alkenyl or a C₂-C₆-alkinyl group; wherein R^{c} refers to a H, a C₁-C₆-alkyl, a C₂-C₆-alkenyl or a C₂-C₆-alkinyl group; wherein Rd refers to a H, a C₁-C₆-alkyl, a C₂-C₆-alkenyl or a C₂-C₆-alkinyl group and Ya refers to a direct binding, a C₁-C₆-alkylen, a C₂-C₆-alkenylen or a C₂-C₆-alkinylen group, wherein each heteroalkyl group can be replace by a carbon atom and one or several hydrogen atoms can be replaced by fluorine or chlorine atoms. Examples of heteroalkyl groups are methoxy, trifluormethoxy, ethoxy, n-propyloxy, iso-propyloxy, tert-butyloxy, methoxymethyl, ethoxymethyl, methoxyethyl, methylamino, ethylamino, dimethylamino, diethylamino, iso-propylethylamino, methyl-aminomethyl, ethylaminomethyl, di-iso-propylaminoethyl, enolether, dimethylaminomethyl, dimethylaminoethyl, acetyl, propionyl, butyryloxy, acetyloxy, methoxycarbonyl, ethoxy-carbonyl, N-ethyl-N-methylcarbamoyl or N-methylcarbamoyl. Other examples of heteroalkyl groups are nitrile, isonitrile, cyanate, thiocyanate, isocyanate, isothiocyanate and alkylnitrile groups.

The term acyl refers to a group of formula —C(═O)-alkyl, —C(═O)-alkenyl or —C(═O)-alkynyl; preferably to a group of formula —C(═O)-alkyl or —C(═O)-alkenyl; especially preferably to a group of formula —C(═O)-alkyl.

The term cycloalkyl refers to a saturated or partially unsaturated (e.g. cycloalkenyl) optionally substituted cyclic group, comprising one or several rings, preferentially one or two rings, containing three to fourteen ring carbon atoms, preferentially three to ten, preferentially three, four, five, six or seven ring carbon atoms. Furthermore the term cycloalkyl refers to a group where one or more hydrogen atoms are replaced by F, Cl, Br, I, OH, ═O , SH, ═S, NH₂, ═NH, or NO₂, or cyclic ketones, for example cyclohexanone, 2-cyclohexenone or cyclopentanone. Examples of cycloalkyl groups are cyclopropyl, cyclobutyl, cyclopentenyl, spiro[4,5]-decanyl, norbornyl, cyclohexyl, cyclopentenyl, cyclohexadienyl, decalinyl, cubanyl, bicyclo[4.3.0]nonyl, tetralin, cyclopentylcyclohexyl, fluorcyclohexyl or the cyclohex-2-enyl group.

The term heterocycloalkyl refers to a cycloalkyl as defined above, wherein one or several, preferentially one, two or three ring carbon atoms are replaced by an O, N, Si, Se, P, S, SO or SO₂, preferentially O, S or N. Preferentially a heterocycloalkyl group is composed of one or two rings comprising three to ten, preferentially three, four, five, six or seven ring atoms. Moreover, the term heterocycloalkyl refers to groups where one or several hydrogen atoms are replaced by F, Cl, Br, I, OH, ═O, SH, ═S, NH₂ or NO₂. Examples of heterocycloalkyl are piperidyl, morpholinyl, urotropinyl, pyrrolidinyl, tetrahydrothiophenyl, tetrahydropyranyl, tetrahydro-furyl, oxacyclopropyl, azacyclopropyl or 2-pyrazolinyl groups as well as lactams, lactons, cyclic imides and cyclic anhydrides.

The term alkylcycloalkyl refers to groups, which contain cycloalkyl as well as alkyl, alkenyl or alkinyl groups according to the above definition, e.g. alkylcycloalkyl, alkylcycloalkenyl, alkenylcycloalkyl and alkinylcycloalkyl groups. Preferentially an alkylcycloalkyl group is composed of a cycloalkyl group, comprising one or more rings, comprising three to ten, preferentially three, four, five, six or seven carbon atoms and one or two alkyl, alkenyl oder alkinyl groups with one or two to six carbon atoms.

The term heteroalkylcycloalkyl refers to alkylcycloalkyl groups, according to the above definition, wherein one or several, preferentially one, two or three carbon atoms are replaced by O, N, Si, Se, P, S, SO or SO₂, preferentialy O, S or N. Preferentially it is composed of one or two ring systems with three to ten, preferentially three, four, five, six or seven ring atoms and one or two alkyl, alkenyl, alkinyl or heteroalkyl groups with one or two to six carbon atoms. Examples of such a group are alkylheterocycloalkyl, alkylheterocycloalkenyl, alkenylheterocycloalkyl, heterocycloalkyl, heteroalkylcycloalkyl, heteroalkylheterocycloalkyl and heteroalkylheterocylcloalkenyl, wherein the cyclic group is saturated or partially (simply, twofold or threefold) unsaturated.

The term aryl or ar refers to an optionally substituted aromatic group, composed of one or several rings, comprising six to fourteen carbon atoms, preferentially six to ten, preferentially six carbon atoms. The term aryl or ar can also refer to an aromatic group, wherein one or several H atoms are replaced by F, Cl, Br or I or OH, SH, NH₂, or NO₂. Examples are phenyl-, naphthyl-, biphenyl-, 2-fluorphenyl, anilinyl-, 3-nitrophenyl or 4-hydroxy-phenyl.

The term heteroaryl refers to an aromatic group, composed of one or several rings, comprising five to fourteen ring atoms, preferentially five to ten, whereof one or several, preferentially one, two, three or four are O, N, P or S ring atoms, preferentially O, S or N. The term heteroaryl can also refer to groups, wherein one or several H atoms are replaced by F, Cl, Br or I or OH, SH, NH₂, or NO₂. Examples are 4-pyridyl, 2-imidazolyl, 3-phenylpyrrolyl, thiazolyl, oxazolyl, triazolyl, tetrazolyl, isoxazolyl, indazolyl, indolyl, benzimidazolyl, pyridazinyl, chinolinyl, purinyl, carbazolyl, acridinyl, pyrimidyl, 2,3″-bifuryl, 3-pyrazolyl and isochinolinyl.

The term aralkyl (or arylalkyl or alkylaryl) refers to groups composed of aryl and alkyl, alkenyl, alkinyl and/or cycloalkyl, e.g. arylalkyl, arylalkenyl, arylalkinyl, arylcycloalkyl, arylcycloalkenyl, alkylarylacycloalkyl and alkylarylcycloalkenyl. Examples of aralkyles are toluol, xylol, mesitylen, styren, benzylchloride, o-fluortoluene, 1H-inden, tetralin, dihydronaphthaline, indanon, phenyl-cyclopentyl, cumol, cyclo-hexylphenyl, fluoren and indan. Preferentially, an aralkyl group is composed of one or two aromatic rings, comprising six to ten ring carbon atoms and one or two alkyl, alkenyl and/or alkinyl comprising one or two to six carbon atoms and/or one cyclo-alkyl comprising five or six ring carbon atoms.

The term heteroaralkyl (or heteroarylalkyl or heteroalkylaryl) refers to an aralkyl group as defined above, wherein one or several, preferentially one, two, three or four carbon atoms are replaced by O, N, Si, Se, P, B or S, preferentially O, N or S, and to groups which contain aryl, heteroaryl and alkyl, alkenyl, alkinyl and/or heteroalkyl and/or cycloalkyl and/or heterocycloalkyl. Preferentially a heteroaralkyl group is composed of one or two aromatic ring systems comprising five or six to ten carbon atoms and one or two alkyl, alkenyl and/or alkinyl comprising one or two to six carbon atoms and/or one cycloalkyl comprising five or six ring carbon atoms, wherein one, two, three or four carbon atoms can be replaced by O, N or S.

Examples are arylheteroalkyl, arylheterocycloalkyl, arylheterocycloalkenyl, arylalkylheterocycloalkyl, arylalkenyl-heterocycloalkyl, arylalkinylheterocyclo-alkyl, arylalkyl-heterocycloalkenyl, heteroarylalkyl, heteroarylalkenyl, heteroarylalkinyl, heteroarylheteroalkyl, heteroarylcyclo-alkyl, heteroarylcycloalkenyl, heteroarylheterocycloalkyl, heteroarylheterocycloalken-yl, heteroarylalkylcycloalkyl, heteroarylalkylheterocycloalkenyl, heteroarylheteroalkylcyclo-alkyl, heteroarylheteroalkylcycloalkenyl and heteroarylhetero-alkyl heterocycloalkyl, wherein the cyclic groups can be saturated or once, twice, three fold of four fold unsaturated. Examples are tetrahydroisochinolinyl, benzoyl, 2- or 3-ethyl-indolyl, 4-methylpyridino, 2-, 3- or 4-methoxyphenyl, 4-ethoxyphenyl, 2-, 3- or 4-carboxyphenylalkyl.

The terms cycloalkyl, heterocycloalkyl, alkylcyclo-alkyl, heteroalkylcycloalkyl, aryl, heteroaryl, aralkyl and heteroaralkyl also refer to groups, wherein one or several H atoms are replaced by F, Cl, Br or I or ═O, OH, SH, NH₂ or NO₂.

The term “optionally substituted” relates to groups, wherein one or several H atoms can be replaced by F, Cl, Br or I or OH, ═O, SH, ═S, NH₂, ═NH, or NO₂. This term relates further to groups, which can be exclusively or additionally substituted with (preferably unsubstituted) C₁-C₆ alkyl, C₂-C₆ alkenyl, C₂-C₆ alkinyl, C₁-C₆ heteroalkyl, C₃-C₁₀ cycloalkyl, C₂-C₉ heterocycloalkyl, C₆-C₁₀ aryl, C₁-C₉ heteroaryl, C₇-C₁₂ aralkyl or C₂-C₁₁ heteroaralkyl groups.

All peptides defined herein can be synthesized from building blocks that can be linked by conducting well established peptide synthesis strategies, e.g. solid-phase peptide synthesis (SPPS) or liquid-phase peptide synthesis (LPPS), using known coupling reagents, e.g. hydroxybenzotriazole (HOBt) and diisopropylcarbodiimide (DIC) or dicyclohexylcarbodiimide (DCC); and known protecting groups and protecting strategies. Unless otherwise defined, all residues are defined as herein.

Protecting groups are known to a person skilled in the art and described in P. J. Kocienski, Protecting Groups, Georg Thieme Verlag, Stuttgart, 1994 and in T. W. Greene, P. G. M. Wuts, Protective Groups in Organic Synthesis, John Wiley & Sons, New York, 1999. Common amino protecting groups are, for instance, t-butyloxycarbonyl (Boc), benzyloxycarbonyl (Cbz, Z), benzyl (Bn), benzoyl (Bz), fluorenylmethyloxycarbonyl (Fmoc), allyloxycarbonyl (Alloc), trichlorethyloxycarbonyl (Trac), acetyl or trifluoracetyl.

Tubulysines and derivatives thereof are known to a person skilled in the art and can e.g. be prepared as described in WO 2008/138561, WO 2004046170, WO 2004/005327, WO 2011/057806, WO 2011/057805 and documents cited therein.

### EXAMPLES

The following derivatives were synthesized from building blocks Z′, L′ and Pep′. The building blocks were synthesized according to methods known to a person skilled in the art.

### Automated Solid Phase Peptide Synthesis of Pep

The peptide moieties (Pep′) of the peptide-drug conjugates Z-L-Pep (formula III) were synthesized according to the Fmoc/tBu protection strategy using an automated multiple solid-phase peptide synthesizer Syro II (MultiSynTech GmbH, Bochum, Gelmany). To gain C-terminal peptide amides, a Rink amide resin with a loading capacity of 0.63 mmol/g was used.

The stepwise synthesis of the complete peptide chains from building blocks is a perseverative cycle of few reactions, i.e. N^{α}-deprotection, amino acid coupling, and some washing steps. In brief, prior to each single amino acid coupling step the base-labile N^{α}-protecting group Fmoc had to be cleaved off from the building blocks, and in a first step from the Rink amide resin as well. For Fmoc cleavage, 400 μL piperidine in DMF (40% v/v) were added to the resin and incubated for 3 min while stirring. The deprotection was repeated with 400 μL piperidine in DMF (20% v/v) for 10 min. Subsequently, the resin was washed with 433 600 μL DMF.

Amino acids were coupled by preincubation of the resin with 200 μL amino acid building block solution (0.5 M in DMF) and 100 μL 3 M Oxyma in DMF for 2 min. Subsequently, 100 μL 3.3 M DIC in DMF were added and the reaction was allowed to proceed for 40 min while stirring. After a washing step with 800 μL DMF, the coupling step was repeated once for each amino acid.

The synthesis of branched peptides was realized by amino acid coupling and sequence elongation, thus sequence branching, at the N^{ε} of lysine. To allow its selective deprotection, a lysine building block with Dde-protected N^{ε} was used. For the selective deprotection of a Dde-protected lysine residue, the fully protected, resin-bound peptide was incubated 12×10 min with 1 mL freshly prepared 3% hydrazine in DMF. After each of the 12 steps, the resin was washed with DMF. Finally, the success of the Dde deprotection was checked by measuring the absorption of the removed hydrazine solution at 301 nm against a reference of fresh hydrazine in DMF. The Dde deprotection was completed if the absorption was <0.1. Otherwise, some more cycles of hydrazine treatment and washing had to be conducted.

### Analytical and Preparative Peptide (Pep′) Cleavage from Resin

For analytical purposes, small amounts of newly synthesized peptides were cleaved off from the resin. Therefore, a small amount of peptide-loaded resin was incubated with TFA/thioanisole/1,2-ethanedithiol (900:70:30 v/v) for 3 h at room temperature, removing all acid-labile protecting groups. Subsequently, the peptide was precipitated for 20 min at −20° C. in 1 mL ice cold diethyl ether, collected by centrifugation (2 min at 7,000 g), and washed with ice cold diethyl ether at least five times. The peptide pellet was dried and finally dissolved in 100 μL H₂O/tBuOH (1:3 v/v) for analysis.

For preparative cleavage, the complete resin was treated as described above. However, precipitation was done in 10 mL ice cold diethyl ether and centrifugation was performed at 4,400 g. The peptide was dried by using a SpeedVac, and finally lyophilized from 1-2 mL H₂O/tBuOH (1:3 v/v).

### Analytical RP-HPLC of Pep′

The synthesized peptides' purity was analyzed by using analytical RP-HPLC on a reversed phase Phenomenex Jupiter Proteo C18 column (4.6 mm×250 mm, 5 μm), and an elution system composed of (A) 0.1% TFA in H₂O and (B) 0.08% TFA in. A linear gradient of 20-70% solvent B in A over 40 min with a flow rate of 0.6 mL min−1 was used. The peptides were detected at 220 nm.

### Preparative RP-HPLC of Pep′

Purification of the synthesized peptides was achieved by preparative RP-HPLC on a Phenomenex Jupiter Proteo C18 column (21.2 mm×250 mm) using an elution system composed of (A) 0.1% TFA in H₂O and (B) 0.08% TFA in ACN, and an appropriate linear gradient of solvent B in A over 40-50 min and a flow rate of 10 mL min−1. For peptide detection, absorption at 220 nm was measured. Fractions were taken and analyzed by MALDI-TOF and/or ESI mass spectrometry and analytical RP-HPLC. Peptide fractions identified to be pure were combined and lyophilized.

### MALDI-TOF Mass Spectrometry of Pep′

For mass analysis using MALDI-TOF mass spectrometry, a matrix consisting of 2,5-dihydroxybenzoic acid and 2-hydroxy-5-methoxybenzoic acid (10 g/L in ACN/H20/TFA 50:49.7:0.3 v/v) was used. The MALDI measurements were conducted by using a Bruker Daltonis Ultraflex III TOF/TOF.

### ESI Ion Trap Mass Spectrometry of Pep′

For mass analysis using ESI Ion Trap mass spectrometry, the samples were diluted to 20 μM in H₂O (0.1% HCOOH) with ACN (7:3 v/v), injected and analyzed. The ESI measurements were conducted by using a Bruker HCT mass spectrometer.

### Commercial Peptide (Pep′) Supply

Alternatively to the aforementioned described in-house synthesis, processing and analysis of the peptide moieties, these peptides were also purchased from established commercial suppliers (e.g. AmbioPharm Inc., North Augusta, S.C., USA).

### Pep1 (OC561): [K4(C-betaA),F7,A33,P34,A35,A36]-pNPY-amide

H-Tyr¹-Pro²-Ser³-Lys⁴(H-Cys-betaAla)-Pros-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Ala³³-Pro³⁴-Ala³⁵-Ala³⁶-NH₂

Calculated average molecular mass: 4167.613 Molecular formula: C189H281N49O56S MS-ESI: 1042.8 [M+4H]⁴⁺

### Pep2 (OC562): [K4(Pam-C-betaA),F7,A33,P34,A35,A36]-pNPY-amide

H-Tyr¹-Pro²-Ser³-Lys⁴(Palmitoyl-Cys-betaAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Ala³³-Pro³⁴-Ala³⁵-Ala³⁶-NH₂

Calculated average molecular mass: 4406.022 Molecular formula: C205H311N49O57S MS-EST: 1100.5 [M−4H]⁴⁻

### Pep3 (OC575): Ac-[K4 (Pam-C-betaA),F7,A33,P34,A35,A36]-pNPY-amide

Acetyl-Tyr¹-Pro²-Ser³-Lys⁴(Palmitoyl-Cys -betaAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Ala³³-Pro³⁴-Ala³⁵-Ala³⁶-NH₂

Calculated average molecular mass: 4448.059 Molecular formula: C207H313N49O58S MS-ESI: 1112.8 [M+4H]⁴⁺

### Pep5 (OC577): [K4 (Pam-C-betaA),F7,A33,P34]-pNPY-amide

H-Tyr¹-Pro²-Ser³-Lys⁴(Palmitoyl-Cys-betaAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-HiS²⁶-Tyr²⁷-Ile²⁸-Asn²⁹-Leu³⁰-Ile³¹-Thr³²-Ala³³-Pro³⁴-Arg³⁵-Tyr³⁶-NH₂

Calculated average molecular mass: 4583.225 Molecular formula: C214H322N52O58S MS-ESI: 1146.7 [M+4H]⁴⁺

### Pep6 (OC579): [K4 (Pam-C-betaA),F7,P34,A35]-pNPY-amide

H-Tyr¹-Pro²-Ser³-Lys⁴(Palmitoyl-Cys-betaAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Arg³³-Pro³⁴-Ala³⁵-Tyr³⁶-NH₂

Calculated average molecular mass: 4583.225 Molecular formula: C214H322N52O58S MS-ESI: 1147.1 [M+4H]⁴⁺

### Pep7 (OC580) : [K4 (Pam-C-betaA),F7,P34,A36]-pNPY-amide

H-Tyr¹-Pro²-Ser³-Lys⁴(Palmitoyl-Cys-betaAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Arg³³-Pro³⁴-Arg³⁵-Ala³⁶-NH₂

Calculated average molecular mass: 4576.238 Molecular formula: C211H325N55O57S MS-ESI: 1144.9 [M+41H]⁴⁺

### Pep8 (OC581): [K4 (Pam-C-betaA), F7,A33,P34,A35]-pNPY-amide

H-Tyr¹-Pro²-Ser³-Lys⁴(Palmitoyl-Cys-betaAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Ala³³-Pro³⁴-Ala³⁵-Tyr³⁶ -NH₂

Calculated average molecular mass: 4498.117 Molecular formula: C211H315N49O58 S MS-ESI: 1125.2 [M+4H]⁴⁺

### Pep9 (OC582): [K4 (Pam-C-betaA),F7,Nle33,P34,Nle35,Nle36]-pNPY-amide

H-Tyr¹-Pro²-Ser³-Lys⁴(Palmitoyl-Cys-betaAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Nle³³-Pro³⁴-Nle³⁵-Nle³⁶-NH₂

(Nle=norleucine)

Calculated average molecular mass: 4532.261 Molecular formula: C214H329N49O57S MS-ESI: 1134.2 [M+4H]⁴⁺

### Pep10 (OC583): [K4 (Pam-C-betaA),F7,Nva33,P34,Nva35,Nva36]-pNPY-amide

H-Tyr¹-Pro²-Ser³-Lys⁴(Palmitoyl-Cys-betaAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁸-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-Nva³³-Pro³⁴-Nva³⁵-Nva³⁶-NH₂

(Nva=norvaline)

Calculated average molecular mass: 4490.181 Molecular formula: C211H323N49O57S MS-ESI: 1122.2 [M+4H]⁴⁺

### Pep11 (OC584): [K4(Pam-C-betaA),F7,NMeA33,P34,NMeA35,NMeA36]-pNPY-amide

H-Tyr¹-Pro²-Ser³-Lys⁴(Palmitoyl-Cys-betaAla)-Pro⁵-Asp⁶-Phe⁷-Pro⁸-Gly⁹-Glu¹⁰-Asp¹¹-Ala¹²-Pro¹³-Ala¹⁴-Glu¹⁵-Asp¹⁶-Leu¹⁷-Ala¹⁸-Arg¹⁹-Tyr²⁰-Tyr²¹-Ser²²-Ala²³-Leu²⁴-Arg²⁵-His²⁶-Tyr²⁷-Ile²⁸-Asn²⁹-Leu³⁰-Ile³¹-Thr³²-NMeAla³³-Pro³⁴-NMeAla³⁶-NMeAla³⁶-NH₂

(NMeA=N-methyl alanine)

Calculated average molecular mass: 4448.102 Molecular formula: C208H317N49O57S MS-ESI: 1112.3 [M+4H]⁴⁺

### Payload (Z′) Supply

Building blocks composed of the payloads (Z′) and the linker structures (L′) of the peptide-drug conjugates Z-L-Pep (formula III) were received from commercial suppliers. For instance, tubulysin derivative building blocks were purchased from TUBE Pharmaceuticals GmbH (Vienna, Austria).

### TubA: Tubulysin A Dithiopyridine Linker (N-[2-(pyridine-2-yldisulfanyl)ethyl]-Tubulysin A)

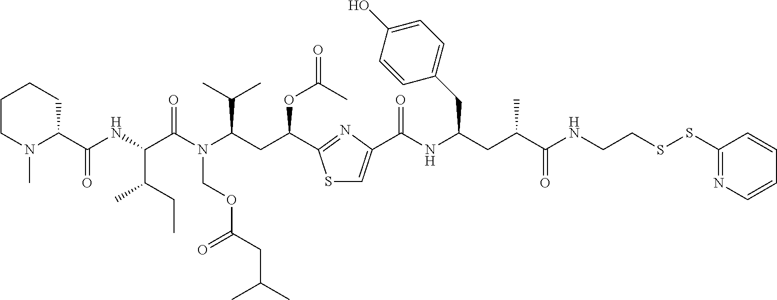 [SMILES: CC[C@H](C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)NCCSSc2ccccn2)cs1)C(C)C]

### Peptide-Drug Conjugates (Z-L-Pep)

### OC563: [K4(Pam-C(TubA)-betaA),F7,A33,P34,A35,A36]-pNPY-amide

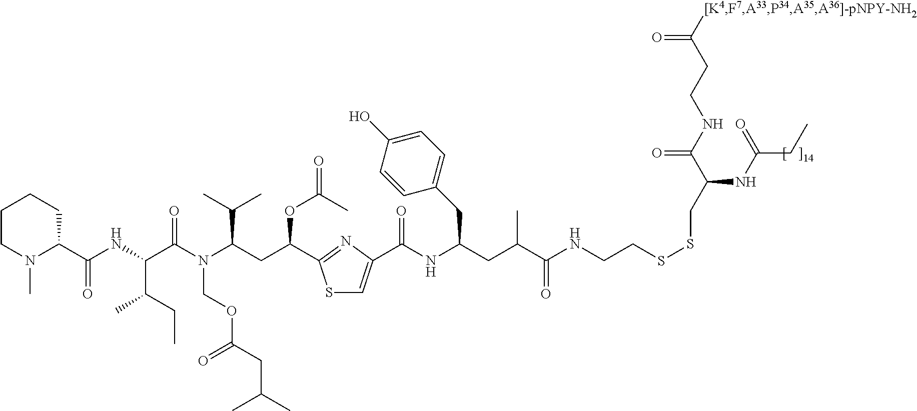

Calculated average molecular mass: 5307.208 Molecular formula: C250H379N55O66S3 MS-ESI: 1327.8 [M+4H]⁴⁺; MS-TOF: 5304.3 [M+H]⁺

### OC591: Ac-[K4 (Pam-C (TubA)-betaA),F7,A33,P34,A35,A36]-pNPY-amide

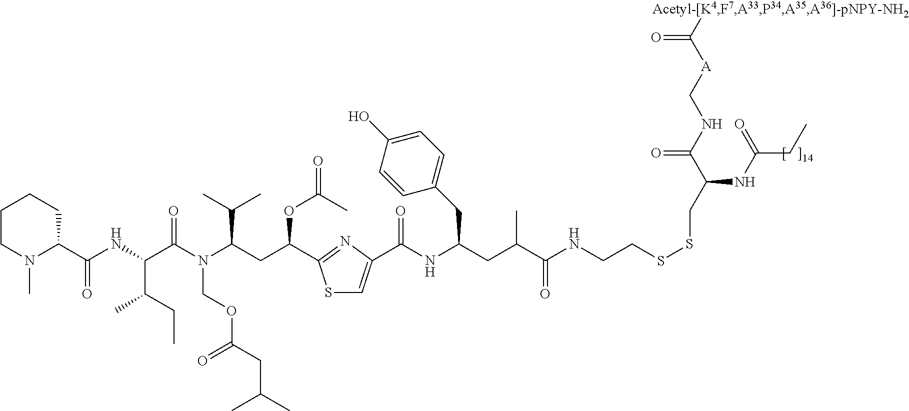

Calculated average molecular mass: 5371.420 Molecular formula: C252H403N55O67S3 MS-ESI: 1342.4 [M+4H]⁴⁺

### OC592: [K4(Pam-C(TubA)-betaA),F7,A33,P34]-pNPY-amide

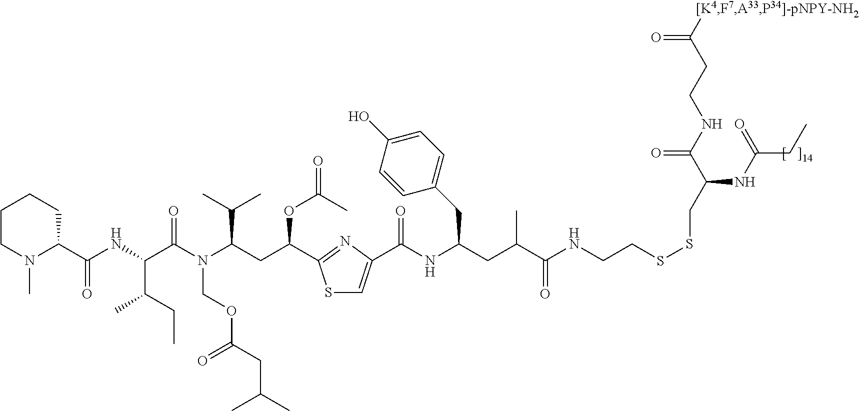

Calculated average molecular mass: 5506.586 Molecular formula: C259H412N58O67S3 MS-ESI: 1376.3 [M+4H]⁴⁺

### OC593: [K4(Pam-C(TubA)-betaA),F7,P34,A35]-pNPY-amide

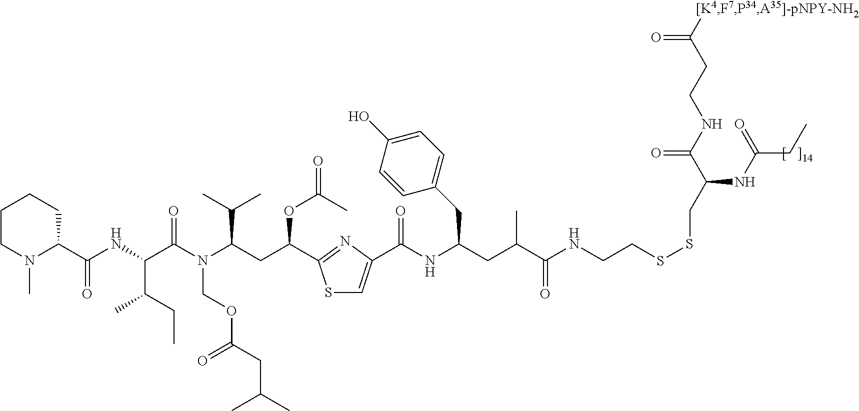

Calculated average molecular mass: 5506.586 Molecular formula: C259H412N58O67S3 MS-ESI: 1377.0 [M+4H]⁴⁺

### OC594: [K4(Pam-C(TubA)-betaA),F7,P34,A36]-pNPY-amide

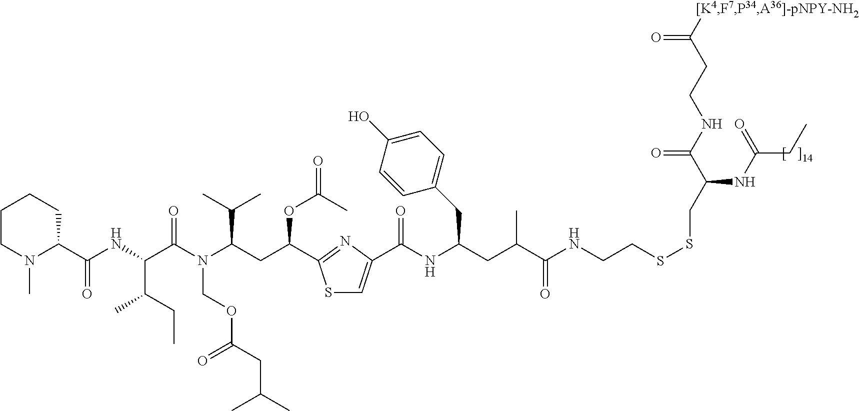

Calculated average molecular mass: 5499.598 Molecular formula: C256H415N61O66S3 MS-ESI: 1376.0 [M+4H]⁴⁺

### OC595: [K4(Pam-C(TubA)-betaA),F7,A33,P34,A35]-pNPY-amide

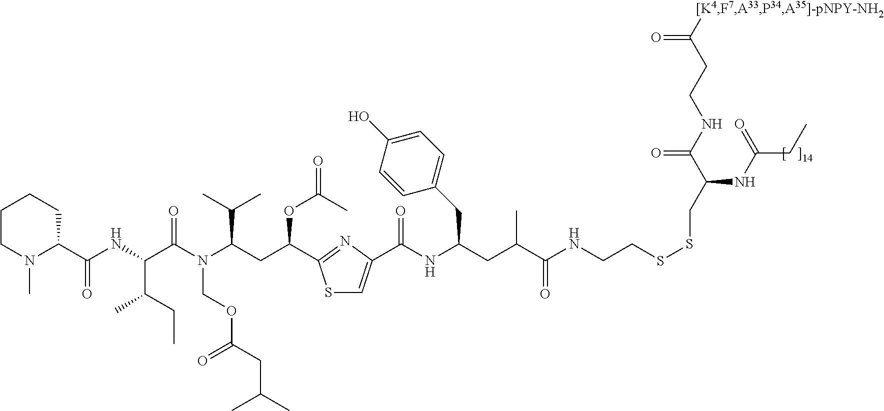

Calculated average molecular mass: 5421.478 Molecular formula: C256H405N55O67S3 MS-ESI: 1356.0 [M+4H]⁴⁺

### OC596: [K4 (Pam-C (TubA) -betaA),F7,Nle33,P34,Nle35,Nle36]-pNPY-amide

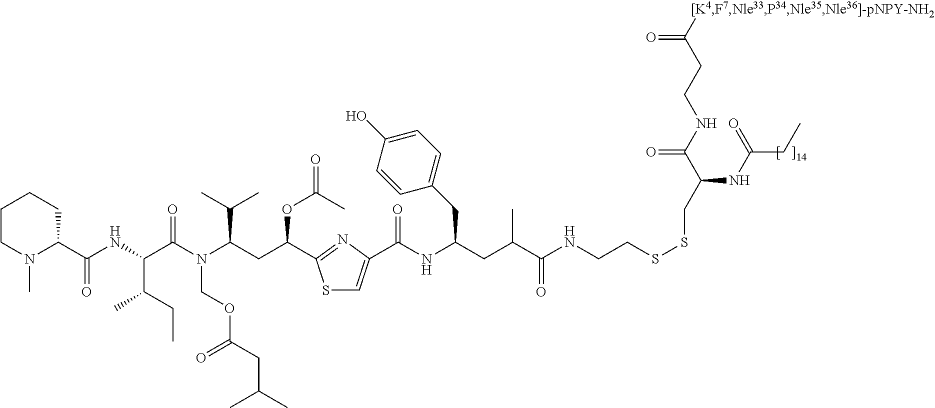

(Nle=norleucine)

Calculated average molecular mass: 5455.622 Molecular formula: C259H419N55O6653 MS-ESI: 1364.7 [M+4H]⁴⁺

### OC597: [K4(Pam-C(TubA)-betaA),F7,Nva33,P34,Nva35,Nva36]-pNPY-amide

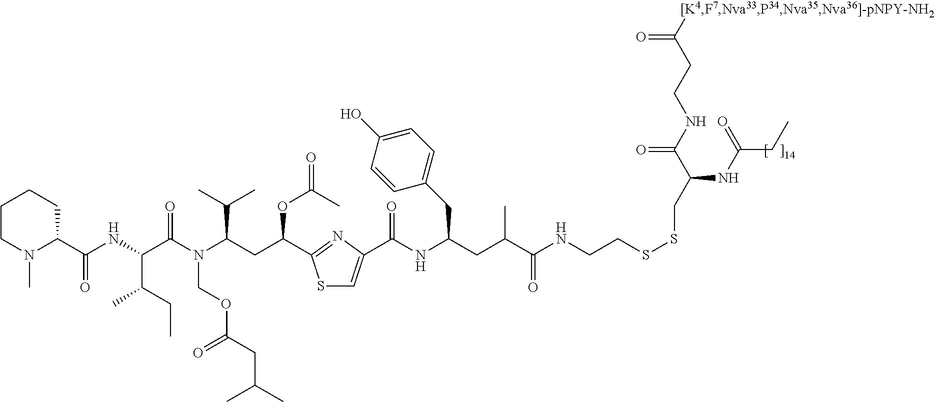

(Nva=norvaline)

Calculated average molecular mass: 5413.542 Molecular formula: C256H413N55O66S3 MS-ESI: 1354.4 [M+4H]⁴⁺

### OC598: [K4(Pam-C(TubA)-betaA),F7,NMeA33,P34,NMeA35,NMeA36]-pNPY-amide

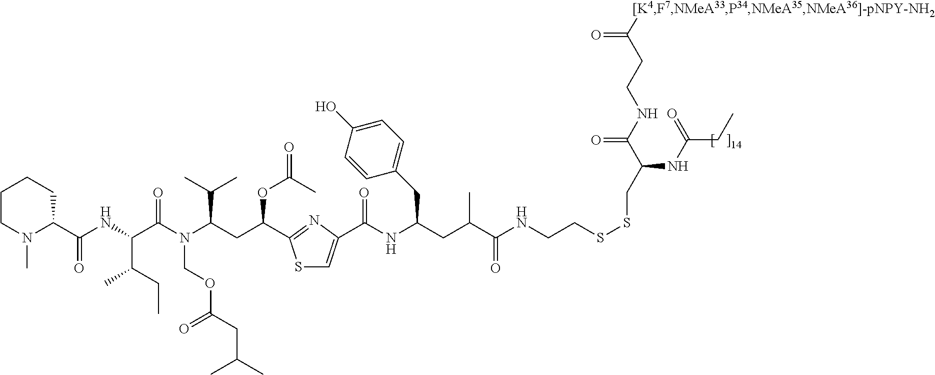

(NMeA=N-methyl alanine)

Calculated average molecular mass: 5371.463 Molecular formula: C253H407N55O66S3 MS-ESI: 1344.0 [M+4H]⁴⁺

### Functional Receptor Activation (Signal Transduction)

The NPY-derived peptide-drug conjugates' ability to

functionally activate the human neuropeptide Y Y1 receptor (hY1R; NPY1R) with high specificity was evaluated by using functional reporter gene assays (using cAMP response element—CRE). For these in vitro assays CHO cells were transiently co-transfected with cDNA encoding human Y1, Y2, Y4, and Y5 receptors, respectively, C-terminally fused to EYFP and the CRE reporter vector pGL4.29 (Promega GmbH, Mannheim, Germany). For this purpose, 2.5.10⁶ CHO cells were seeded per 25 cm² cell culture flask and allowed to adhere overnight. Subsequently, co-transfection of the cells was done using 10 μg hYxR vector, 2 μg pGL4.29 reporter vector and 25 μL of Metafectene® Pro transfection reagent (Biontex Laboratories GmbH, Martinsried, Germany) per culture flask. After 3 hours transfection in OptiMEM under standard growth conditions, the transfection solution was discarded, transfected cells were detached and seeded in white/clear bottom 96-well plates (50,000 cells/well). The cells were cultured for 48 hours under standard growth conditions to facilitate receptor and reporter gene expression. Subsequently, the transfected cells were co-stimulated with 10⁻⁶ M forskolin (adenylyl cyclase activator for cAMP elevation) and 10⁻¹¹-10⁻⁶ M of peptide-drug conjugates under investigation (reduction of cAMP levels by Gαi-mediated signal transduction of activated hYx receptors). After 6 hours stimulation at 37° C., incubation media were removed and 60 μL/96-well of Promega's ONE-Glo™ reagent (1:1 in DMEM/Ham's F-12, v/v) were added. After 10 min incubation at room temperature the reporter gene generated luminescence signal was measured by using a Synergy 2 multiwell plate reader (BioTek, Bad Friedrichshall, Germany).

FIG. 1 shows EC₅₀ curves and values of the functional activation of the human NPY Y1 receptor, compared to the human Y4 receptor, by the peptide-drug conjugate OC563 as determined by CRE reporter gene assays.

### In Vitro Efficacy

For early in vitro evaluations of the anti-proliferative and cytotoxic effects, respectively, of the peptide-drug conjugates of formula Z-L-Pep, a fluorometric resazurin-based cell proliferation/viability assay was used. Human cancer cell lines (primarily breast cancer-derived and the Ewing's sarcoma cell line SK-N-MC) and non-cancer cell lines were seeded with low densities into 96-well plates (1,500-20,000 cells per well), and were allowed to adhere for 24 h. Subsequently, the compounds—dissolved to appropriate concentrations in cell line-specific medium—were added to the cells and incubated for 2-72 or 96 h, respectively. In case the initial compound treatment was shorter than 72 or 96 h, respectively, the incubation solution was discarded, cells were rinsed once with cell culture medium and were allowed to proliferate in compound-free medium until 72 or 96 h, respectively, were reached. Subsequently, medium was replaced by 50 μM resazurin in medium, and the cells were incubated for 2 h. Finally, the conversion of resazurin to resorufin by viable, metabolically active cells was measured using a Synergy 2 multiwell plate reader (BioTek, Bad Friedrichshall, Germany) with 540 nm excitation and 590 nm emission filter setting. Dose-response curves were analyzed by using GraphPad Prism 5.04 resulting in IC₅₀ values.

FIG. 2 shows the inhibition of the cell proliferation of various breast cancer cell lines (MCF-7, T-47D, MDA-MB-468) and the Ewing's sarcoma cell line SK-N-MC resulting from initial 6 h treatment with peptide-drug conjugate OC563. IC₅₀ values were calculated by using GraphPad Prism 5.04 based on the depicted dose-response curves. OC563 caused a strong anti-proliferative and cytotoxic effect that correlated very well with the NPY Y1 receptor expression in the different cell lines, since the order of Y1 receptor expression levels was determined by quantitative real-time PCR to be as follows (from high expression to lower expression): SK-N-MC>MCF-7>T-47D>MDA-MB-468.

### Peptide-Drug Conjugate (Z-L-Pep)-Induced Receptor Internalization

As the specific receptor-mediated internalization of the peptide-drug conjugates into the characteristically receptor-(over)expressing diseased cells is the major prerequisite for the aspired targeted therapy, the efficacy of the peptide-drug conjugate-induced receptor internalization was tested by conducting in vitro fluorescence microscopy studies. For that purpose, 2.5·10⁶ CHO cells were seeded per 25 cm² cell culture flask and allowed to adhere overnight. Then, the cells were transiently transfected with cDNA encoding human NPY Y1 receptor that was C-terminally fused to EYFP. The transfection mix contained 10 μg receptor vector and 25 μL Lipofectamine® 2000 transfection reagent (Thermo Fisher Scientific, Waltham, Mass., USA) in 6 mL OptiMEM, and was incubated with the cells for 6 h under standard growth conditions. Subsequently, the transfection solution was discarded, the transfected cells were detached from the culture flask and seeded in Falcon® 8-well chamber slides (Corning, Corning, N.Y., USA) (50,000 cells/well). The cells were cultured for 16 hours in DMEM/Ham's F-12 under standard growth conditions to facilitate receptor expression. Subsequently, the transfected cells were rinsed once with PBS, starved for 30 min with OptiMEM, and then stimulated with 10⁻⁶ M peptide-drug conjugate in OptiMEM for 1 h under standard growth conditions. Subsequently, the cells were rinsed three times with ice-cold PBS, the nuclei were dyed with Hoechst 33342 (0.5 mg/mL), followed by further washing cycles with ice-cold PBS. Finally, the unfixed cells (to avoid fixation artifacts) were covered by Fluoromount-G mounting medium (SouthernBiotech, Birmingham, Ala., USA) and immediately inspected by using a laser scanning microscope LSM 700 or an Axio Observer microscope equipped with an ApoTome imaging system (both: Zeiss, Jena, Germany).

FIG. 3A illustrates the localization of the majority of NPY Y1 receptors (visualized by its C-terminal EYGP-tag; pseudo-color dark gray) within the plasma membrane in transiently transfected, but unstimulated CHO cells. However, as shown in FIG. 3B, the cells' stimulation with the NPY Y1 receptor-selective peptide-drug conjugate OC563 resulted in substantial peptide-drug conjugate-induced internalization of the Y1 receptors due to the binding and subsequent activation of the receptor by the ligand, as indicated by the loss of receptors (pseudocolor dark gray) in the membrane and increasing intracellular vesicular spots due to endocytotic receptor internalization.

### In Vivo Efficacy (Study 1—Breast Cancer)

The in vivo efficacy of selected peptide-drug conjugates (Z-L-Pep) was tested by using XenTech's patient-derived breast cancer xenograft (PDX) model T272 (XenTech SAS, Evry, France). Female athymic nude-Foxnlnu (outbred) mice (Envigo, Gannat, France) were 6-7 weeks old when the patient-derived tumor specimens of the T272 model, an ER+/PR+ xenograft derived from breast infiltrating ductal adenocarcinoma, were inoculated. For tumor inoculation, the mice were anaesthetized with 100 mg/kg ketamine hydrochloride and 10 mg/kg xylazine, then the skin was aseptized with chlorhexidine solution, incised at the level of the interscapular region, and a 20 mm³ tumor fragment was placed in the subcutaneous tissue. Finally, the skin was closed with clips.

The mice were housed in groups of a maximum of 5 animals during the experimental phase in individually ventilated cages (IVC) of polysulfone (PSU) plastic (mm 213 W×362 D×185 H; Allentown, USA) with sterilized and dust-free bedding cobs, and under a light-dark cycle (14-hours circadian cycle of artificial light) and controlled room temperature and humidity. Daily, each mouse was offered a complete pellet diet (150-SP-25, SAFE) and filtered, sterilized tap water. T272 tumor-bearing mice received p-estradiol (8.5 mg/L) with the drinking water, from the day of tumor implantation to the end of the study.

Each study group comprised 10 fit mice, each of them with at least 20 g body weight at the day of randomization and inoculation. Treatment started with mean tumor volumes of 110-120 mm³ (range 60-200 mm³). Animals were treated with an application volume of 10 mL/kg by slow i.v. route with 2 mg/kg of the peptide-drug conjugates (Z-L-Pep) tested. As vehicle a physiological (0.9%) NaCl solution with 2.5% ethanol (v/v) was used. Animals were treated three times a week for 3 weeks (D0-D26), followed by a follow-up period of further three weeks (D27-D47). All animals were sacrificed at the end of the experimental phase (D48).

During the whole experimental period, from grafting day to study termination, the mice were observed daily for physical appearance, behaviour, clinical signs and body weight (BW two times a week during the follow-up period). Tumor growth was measured three times a week during the treatment phase and two times a week during the follow-up period. Tumor growth was monitored by calliper measurement and tumor volume was calculated according to the formula W²×L/2, where the length (L) and the width (W) were the longest and the shortest diameters of the tumor, respectively.

FIG. 4 illustrates the in vivo efficacy of the peptide-drug conjugate (Z-L-Pep) OC563, with modified peptide C-terminus in the sense of the present application, compared to two peptide-drug conjugates with the unmodified C-terminus of wild type NPY, OC528 (PCT/EP2013/002790) and OC1508 (PCT/EP2015/000558). The in vivo efficacy was tested in the subcutaneous patient-derived breast cancer xenograft (PDX) model T272 (Xentech SAS, Evry, France). Ten mice per study group were treated by slow i.v. route with 10 mL/kg vehicle (physiological 0.9% NaCl solution with 2.5% ethanol, v/v) and 2 mg/kg of peptide-drug conjugate in vehicle, respectively, three times a week for three weeks (D0-D26), followed by a three weeks follow-up period (D27-D47). The tumor volumes were measured using a caliper and were normalized to the tumor volume at the day of the first treatment (D0), which was set 100, resulting in values of relative tumor volumes (RTVs). FIG. 4A shows the curves of relative tumor volumes for OC563, subject of the present application, compared to the vehicle group as well as groups treated with OC528 and OC1508, respectively. OC563 treatment was significantly more effective than OC528 and OC1508. OC563 reached a T/C % value of 28.3%, which was far better than the best conventional treatment of the T272 model tested so far, according to the supplier's model characterization: a combination of adriamycin (2 mg/kg)/cyclophosphamide (100 mg/kg) with a T/C % value of 42%. FIG. 4B shows the in vivo data as Kaplan-Meier plot representing the median doubling times of the relative tumor volumes. As illustrated, OC563's RTV doubling time is with 44 days more than three times higher than that of untreated tumors (vehicle; 14 days), and more than two and three times higher than the doubling times of OC528 (19.5 days) and OC1508 (13 days), respectively. Furthermore, OC563 effected tumor free survival in 11% of the animals, complete tumor regression (11%), partial tumor regression (22%) and in further 55% of the animals tumor stabilization.

Hence, OC563, subject of the present application, is significantly more anti-tumor effective in vivo than other peptide-drug conjugates with a peptide C-terminus comparable to wild type NPY, as demonstrated with OC528 (PCT/EP2013/002790) and OC1508 (PCT/EP2015/000558).

### In Vivo Efficacy (Study 2—Ewing's Sarcoma)

The in vivo efficacy of the peptide-drug conjugate OC563 was tested by using a patient-derived Ewing's sarcoma xenograft (PDX) model (EPO GmbH, Berlin-Buch, Germany; model Sarc10228). Female NMRI-nu/nu mice were 6-7 weeks old when the patient-derived tumor specimens of the Sarc10228 model, hY1R-overexpressing Ewing's sarcoma, were inoculated.

Each study group comprised 3 fit mice, each of them with at least 20 g body weight at the day of randomization and inoculation. Animals were treated with an application volume of 10 mL/kg by slow i.v. route with 2 mg/kg of the peptide-drug conjugate OC563. As vehicle a physiological (0.9%) NaCl solution with 2.5% ethanol (v/v) was used. Animals were treated three times a week for 3 weeks (D0-D18). All animals were sacrificed at the end of the experimental phase.

During the whole experimental period, from grafting day to study termination, the mice were observed daily for physical appearance, behaviour, clinical signs and body weight. Tumor growth was measured two times a week. Tumor growth was monitored by calliper measurement and tumor volume was calculated according to the formula W²×L/2, where the length (L) and the width (W) were the longest and the shortest diameters of the tumor, respectively.

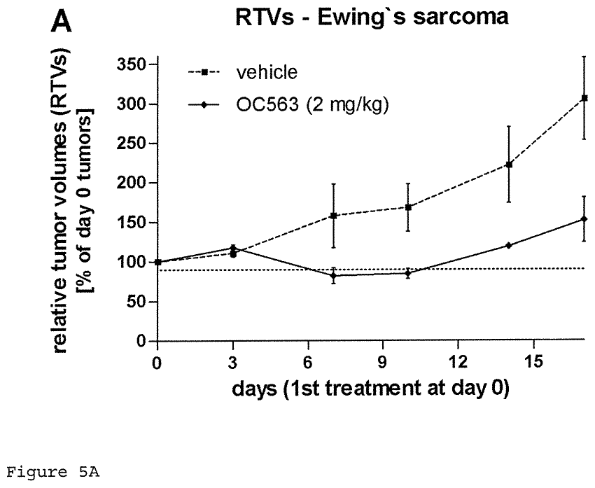

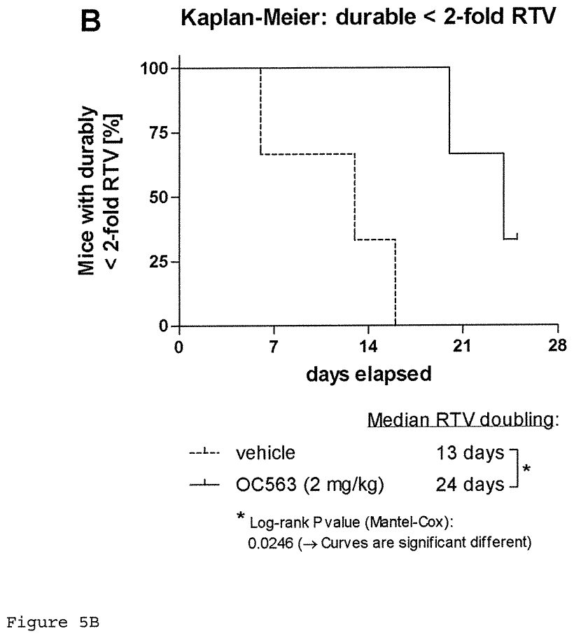

FIG. 5 illustrates the in vivo efficacy of the peptide-drug conjugate (Z-L-Pep) OC563, with modified peptide C-terminus in the sense of the present application. The in vivo efficacy was tested in the subcutaneous patient-derived Ewing's sarcoma xenograft (PDX) model Sarc10228 (EPO GmbH, Berlin-Buch, Germany). Three mice per study group were treated by slow i.v. route with 10 mL/kg vehicle (physiological 0.9% NaCl solution with 2.5% ethanol, v/v) and 2 mg/kg of OC563 in vehicle, respectively, three times a week for three weeks (D0-D18). The tumor volumes were measured using a caliper and were normalized to the tumor volume at the day of the first treatment (D0), which was set 100%, resulting in values of relative tumor volumes (RTVs). FIG. 5A shows the curves of relative tumor volumes for OC563, subject of the present application, compared to the vehicle group. OC563 reached a T/C % value of ˜50%. FIG. 5B shows the in vivo data as Kaplan-Meier plot representing the median doubling times of the relative tumor volumes. As illustrated, OC563's RTV doubling time of 24 days is around two times higher than that of untreated tumors (vehicle; 13 days).

### Data Analysis

For data analysis GraphPad Prism 5.04 and LibreOffice Calc 5.3.3.2 were used.

Surprisingly, as exemplified by compound OC563, a peptide-toxin conjugate comprising a peptide moiety of the present invention permitted good functional hY1R activation and hY1R-mediated internalization in vitro; against all scientific conviction of the NPY receptor community as aforementioned.

Even more surprisingly, PDCs comprising these novel artificially modified peptide moieties with its strongly atypical C-terminus permitted in vitro anti-tumor efficacies in a hY1R expression-level dependent manner with IC50 values in the low nanomolar range.

Very surprisingly, PDCs comprising these novel artificially modified peptide moieties with its strongly atypical C-terminus permitted potent in vivo anti-tumor efficacy in a patient-derived breast cancer xenograft (breast cancer PDX) as well. Most surprisingly, and contrarily to all established conviction on prerequisites for a potent hY1R-addressing peptide, PDCs comprising these novel artificially modified peptide moieties with its strongly atypical C-terminus were significantly more effective in the breast cancer PDX animal models than PDCs containing the well-established “gold standard” of highly affine hY1R-selective peptides, [F⁷,P³⁴]-pNPY (see FIGS. 4A and 4B, wherein the novel conjugate OC563 claimed herein is compared to the recently disclosed OC528 and OC1508; PCT/EP2013/002790 and PCT/EP2015/000558).

1. A compound having the following formula (I):
R¹-Tyr-Pro-Ser-Lys-Pro-Asp-Phe-Pro-Gly-Glu-Asp-Ala-Pro-Ala-Glu-Asp-Leu-Ala-Arg-Tyr-Tyr-Ser-Ala-Leu-Arg-His-Tyr-Ile-Asn-Leu-Ile-Thr-Xaa³³-Pro-Xaa³⁵-Xaa³⁶-NH₂ (I)
wherein
R¹ is hydrogen or an acyl group;
Xaa³³ is Arg or a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is Arg or a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1; and
Xaa³⁶ is Tyr or a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1;
with the proviso that Xaa³³ is not Arg, when Xaa³⁵ is Arg and Xaa³⁶ is Tyr;
or a salt thereof.

2. The compound according to claim 1 having the following formula (I):
R¹-Tyr-Pro-Ser-Lys-Pro-Asp-Phe-Pro-Gly-Glu-Asp-Ala-Pro-Ala-Glu-Asp-Leu-Ala-Arg-Tyr-Tyr-Ser-Ala-Leu-Arg-His-Tyr-Ile-Asn-Leu-Ile-Thr-Xaa³³-Pro-Xaa³⁵-Xaa³⁶-NH₂ (I)
wherein
R¹ is hydrogen or an acyl group;
Xaa³³ is a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1; and
Xaa³⁶ is a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1;
or a salt thereof.

3. A compound having the following formula (II):
R¹-Tyr-Pro-Ser-Lys(14⁸)-Pro-A sp-Phe-Pro-Gly-Glu-A sp-Ala-Pro-Ala-Glu-A sp-Leu-Ala-Arg-Tyr-Tyr-S er-Ala-Leu-Arg-His-Tyr-Ile-Asn-Leu-Ile-Thr-Xaa³³-Pro-Xaa³⁵-Xaa³⁶-NH₂ (II)
wherein
R¹ is hydrogen or an acyl group;
Xaa³³ is Arg or a group of formula —N(R²)—CH(R³)—(CH₂)ₙ—C(50 O)—, wherein R² is hydrogen or a methyl group, R³ is hydrogen or a linear or branched C₁₋₈ alkyl group and n is 0 or 1;
Xaa³⁵ is Arg or a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1;
Xaa³⁶ is Tyr or a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1; and
R⁸ is bound to the nitrogen atom at the side chain of the lysine (NE) and is selected from the following groups: R⁹-Cys- and R⁹-Cys-βAla-, wherein R⁹ is hydrogen or an acyl group;
with the proviso that Xaa³³ is not Arg, when Xaa³⁵ is Arg and Xaa³⁶ is Tyr;
or a salt thereof.

4. The compound according to claim 3 having the following formula (II):
R¹-Tyr-Pro-Ser-Lys(R⁸)-Pro-Asp-Phe-Pro-Gly-Glu-Asp-Ala-Pro-Ala-Glu-Asp-Leu-Ala-Arg-Tyr-Tyr-S er-Ala-Leu-Arg-His-Tyr-Ile-Asn-Leu-Ile-Thr-Xaa³³-Pro-Xaa³⁵-Xaa³⁶-NH₂ (II)
wherein
R¹ is hydrogen or an acyl group;
Xaa³³ is a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1;
Xaa³⁶ is a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1; and
R⁸ is bound to the nitrogen atom at the side chain of the lysine (Nε) and is selected from the following groups: R⁹-Cys- and R⁹-Cys-βAla-, wherein R⁹ is hydrogen or an acyl group;
or a salt thereof.

5. The compound according to claim 1, wherein R¹ is hydrogen or an acetyl group and Xaa³³, Xaa³⁵ and Xaa³⁶ are independently selected from alanine (Ala; A), valine (Val; V), leucine (Leu; L), isoleucine (Ile; I), beta-alanine (βAla; βA), N-methyl-alanine (N-Me-Ala), norvaline (Nva), norleucine (Nle), β-homo-leucine (β-homo-Leu), β-homo-isoleucine β-homo-Ile), N-methyl-isoleucine (N-Me-Ile), and N-methyl-norleucine (N-Me-Nle).

6. A compound according to claim 3, wherein R⁹ is selected from the following groups: palmitoyl, tetradecanoyl, dodecanoyl, decanoyl, octadecanoyl or acetyl; preferably from palmitoyl and dodecanoyl; especially preferably, R⁹ is palmitoyl.

7. A compound according to claim 1, wherein the compound is:
H-Tyr-Pro-Ser-Lys-Pro-Asp-Phe-Pro-Gly-Glu-Asp-Ala-Pro-Ala-Glu-Asp-Leu-Ala-Arg-Tyr-Tyr-Ser-Ala-Leu-Arg-His-Tyr-Ile-Asn-Leu-Ile-Thr-Ala-Pro-Ala-Ala-NH₂; or
Acetyl-Tyr-Pro-Ser-Lys-Pro-Asp-Phe-Pro-Gly-Glu-Asp-Ala-Pro-Ala-Glu-Asp-Leu-Ala-Arg-Tyr-Tyr-Ser-Ala-Leu-Arg-His-Tyr-Ile-Asn-Leu-Ile-Thr-Ala-Pro-Ala-Ala-NH₂;
or a salt thereof.

8. A compound of formula (III):
Pep-L-Z (III)
wherein
Pep is a compound of formula (II′)
R¹-Tyr-Pro-Ser-Lys(R⁸)-Pro-Asp-Phe-Pro-Gly-Glu-Asp-Ala-Pro-Ala-Glu-Asp-Leu-Ala-Arg-Tyr-Tyr-Ser-Ala-Leu-Arg-His-Tyr-Ile-Asn-Leu-Ile-Thr-Xaa³³-Pro-Xaa³⁵-Xaa³⁶-NH₂ (II′)
wherein
R¹ is hydrogen or an acyl group;
Xaa³³ is Arg or a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is Arg or a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1;
Xaa³⁶ is Tyr or a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1;
with the proviso that Xaa³³ is not Arg, when Xaa³⁵ is Arg and Xaa³⁶ is Tyr;
and
R⁸ is bound to the nitrogen atom at the side chain of the lysine (Nε) and is selected from the following groups: R⁹-Cys- and R⁹-Cys-βAla-, wherein R⁹ is hydrogen or an acyl group;
wherein the hydrogen atom at the SH moiety of Cys at group R⁸ is replaced by the bond to L;
L is a linker between Pep and Z; and
Z is a natural or synthetic tubulysin derivative wherein one hydrogen atom or one OH group has been replaced by the bond to L;
or a salt thereof.

9. The compound of formula (III) according to claim 8:
Pep-L-Z (III)
wherein
Pep is a compound of formula (II′)
R¹-Tyr-Pro-Ser-Lys(R⁸)-Pro-A sp-Phe-Pro-Gly-Glu-A sp-Ala-Pro-Ala-Glu-A sp-Leu-Ala-Arg-Tyr-Tyr-S r-Ala-Leu-Arg-His-Tyr-Ile-Asn-Leu-Ile-Thr-Xaa³³-Pro-Xaa'-Xaa³⁶-NH₂ (II′)
wherein
R¹ is hydrogen or an acyl group;
Xaa³³ is a group of formula —N(R²)—CH(R³)—(CH₂)ₙ—C(═O)—, wherein R² is hydrogen or a methyl group, R³ is hydrogen or a linear or branched C₁₋₈ alkyl group and n is 0 or 1;
Xaa³⁵ is a group of formula —N(R⁴)—CH(R⁵)—(CH₂)ₘ—C(═O)—, wherein R⁴ is hydrogen or a methyl group, R⁵ is hydrogen or a linear or branched C₁₋₈ alkyl group and m is 0 or 1;
Xaa³⁶ is a group of formula —N(R⁶)—CH(R⁷)—(CH₂)ₚ—C(═O)—, wherein R⁶ is hydrogen or a methyl group, R⁷ is hydrogen or a linear or branched C₁₋₈ alkyl group and p is 0 or 1; and
R⁸ is bound to the nitrogen atom at the side chain of the lysine (Nε) and is selected from the following groups: R⁹-Cys- and R⁹-Cys-≢Ala-, wherein R⁹ is hydrogen or an acyl group;
wherein the hydrogen atom at the SH moiety of Cys at group R⁸ is replaced by the bond to L;
L is a linker between Pep and Z; and
Z is a natural or synthetic tubulysin derivative wherein one hydrogen atom or one OH group has been replaced by the bond to L;
or a salt thereof.

10. The compound according to claim 8, wherein L is selected from the following groups:
—CH₂—CH₂——;
—O—CH₂—CH₂—S—;
—NH—CH₂—CH₂—S—; or
—NH—NH—C(═O)—O—CH₂—CH₂—S—;
wherein the sulphur of L is bound to the sulphur of the Cys at group R⁸.

11. The compound according to claim 8, wherein Z is a compound of formula (IV): 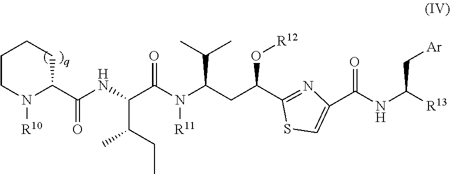
wherein
q is 0, 1 or 2;
R¹° is an alkyl, acyl or a heteroalkyl group;
R¹¹ is an optionally substituted alkyl, alkenyl, alkinyl, acyl, heteroalkyl, cycloalkyl, heterocycloalkyl, alkylcycloalkyl, heteroalkylcycloalkyl, aryl, heteroaryl, aralkyl or heteroaralkyl group;
R¹² is hydrogen or an optionally substituted alkyl, alkenyl, alkinyl, acyl, heteroalkyl, cycloalkyl, heterocycloalkyl, alkylcycloalkyl, heteroalkylcycloalkyl, aryl, heteroaryl, aralkyl or heteroaralkyl group;
R¹³ is a group of formula -COOH, -CONH2, -CONHNH2 or -CH2OH or a group of the following formula: 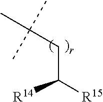
wherein r is 0 or 1; R¹⁴ is hydrogen or an optionally substituted C₁₋₆ alkyl group or an optionally substituted aryl or heteroaryl group; and R¹⁵ is a group of formula —COOH, —CONH₂, —CONHNH₂ or —CH₂OH; and
Ar is an optionally substituted arylene or heteroarylene group;
wherein one OH group of a COOH group or one hydrogen atom has been replaced by the bond to L.

12. The compound according to claim 8, wherein Z has the following formula: 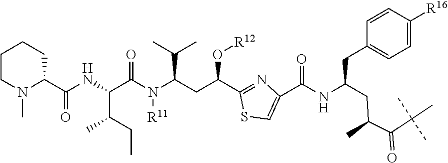
wherein
R11 is hydrogen, a C₁₋₆ alkyl group, or a group of formula —CH₂—O—C(═O)—R¹⁷; wherein R¹⁷ is a C₁₋₆ alkyl group or a C₂₋₆ alkenyl group or an aryl group or a heteroaryl group;
R¹² is a C₁₋₆ alkyl group or an acetyl group; and
R¹⁶ is hydrogen, halogen, OH, NO₂, NH₂, CN, C₁₋₆ alkyl, —O—C₁₋₆ alkyl, phenyl, -NH-C1-6 alkyl or —N(C₁₋₆ alkyl)₂.

13. The compound according to claim 8, wherein Z has the following formula: 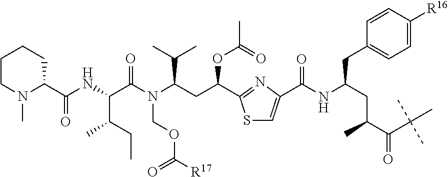
wherein R¹⁷ is hydrogen, or an alkyl, alkenyl, aryl or heteroaryl group and R¹⁶ is hydrogen or a hydroxy group.

14. A pharmaceutical composition containing a compound according to claim 8 and optionally one or more carriers and/or adjuvants.

15. A method of treating a cancer comprising administering a compound according to claim 8 to a subject.

16. The compound according to claim 3, wherein R¹ is hydrogen or an acetyl group and Xaa³³, Xaa³⁵ and Xaa³⁶ are independently selected from alanine (Ala; A), valine (Val; V), leucine (Leu; L), isoleucine (Ile; I), beta-alanine (βAla; βA), N-methyl-alanine (N-Me-Ala), norvaline (Nva), norleucine (Nle), β-homo-leucine (β-homo-Leu), β-homo-isoleucine (β-homo-IIe), N-methyl-isoleucine (N-Me-Ile), and N-methyl-norleucine (N-Me-Nle).

17. A compound according to claim 3, which the compound is:
H-Tyr-Pro-Ser-Lys (Palmitoyl-Cys-(3Ala)-Pro-Asp-Phe-Pro-Gly-Glu-Asp-Ala-Pro-Ala-Glu-Asp-Leu-Ala-Arg-Tyr-Tyr-Ser-Ala-Leu-Arg-His-Tyr-Ile-Asn-Leu-Ile-Thr-Ala-Pro-Ala-Ala-NH₂; or
Acetyl-Tyr-Pro-Ser-Lys (Palmitoyl-Cys-(βAla)-Pro-Asp-Phe-Pro-Gly-Glu-Asp-Ala-Pro-Ala-Glu-Asp-Leu-Ala-Arg-Tyr-Tyr-Ser-Ala-Leu-Arg-His-Tyr-Ile-A sn-Leu-Ile-Thr-Ala-Pro-Ala-Ala-NH₂;
or a salt thereof.


SEQUENCE LISTING (3 sequences)

SEQ ID NO: 1 - protein, 36 aa, Artificial Sequence
  Analogue of Neuropeptide Y
  MISC_FEATURE (33)..(33): Arg or a group of formula -N(R2)-CH(R3)-(CH2)n- C(=O)-, wherein R2 is hydrogen or a methyl group, R3 is hydrogen or a linear or branched C1-8 alkyl group and n is 0 or 1; with the proviso that Arg is excluded when Xaa35 is Arg and Xaa36 is Tyr
  MISC_FEATURE (35)..(35): Arg or a group of formula -N(R4)-CH(R5)-(CH2)m- C(=O)-, wherein R4 is hydrogen or a methyl group, R5 is hydrogen or a linear or branched C1-8 alkyl group and m is 0 or 1
  MISC_FEATURE (36)..(36): Tyr or a group of formula -N(R6)-CH(R7)-(CH2)p- C(=O)-, wherein R6 is hydrogen or a methyl group, R7 is hydrogen or a linear or branched C1-8 alkyl group and p is 0 or 1
YPSKPDFPGEDAPAEDLARYYSALRHYINLITXPXX

SEQ ID NO: 2 - protein, 36 aa, Artificial Sequence; identical to SEQ ID NO: 1
  Analogue of Neuropeptide Y
  MISC_FEATURE (33)..(33): alanine, valine, leucine, isoleucine, beta- alanine, N-methyl-alanine, norvaline, norleucine, beta-homo- leucine, beta-homo-isoleucine, N-methyl-isoleucine, N-methyl- norleucine
  MISC_FEATURE (35)..(35): alanine, valine, leucine, isoleucine, beta- alanine, N-methyl-alanine, norvaline, norleucine, beta-homo- leucine, beta-homo-isoleucine, N-methyl-isoleucine, N-methyl- norleucine
  MISC_FEATURE (36)..(36): alanine, valine, leucine, isoleucine, beta- alanine, N-methyl-alanine, norvaline, norleucine, beta-homo- leucine, beta-homo-isoleucine, N-methyl-isoleucine, N-methyl- norleucine

SEQ ID NO: 3 - protein, 36 aa, Artificial Sequence
  Analogue of Neuropeptide Y
YPSKPDFPGEDAPAEDLARYYSALRHYINLITAPAA